(19) United States
(12) Patent Application Publication
(10) Pub. No.: US 2024/0417701 A1
(43) Pub. Date: Dec. 19, 2024

(54) Method For Improving Mitochondrial Functions

(71) Applicants: Bio-Xcelerator, Inc., Tokyo (JP); TOHOKU UNIVERSITY, Sendai-shi, Miyagi (JP)

(72) Inventor: Takaaki AKAIKE, Sendai-shi (JP)

(21) Appl. No.: 18/740,839

(22) Filed: Jun. 12, 2024

Related U.S. Application Data
(63) Continuation of application No. 17/327,750, filed on May 23, 2021, now abandoned.
(60) Provisional application No. 63/029,467, filed on May 23, 2020.

(51) Int. Cl. C12N 9/02

(52) U.S. Cl. CPC C12N 9/0051; C12Y 108/05

(57) ABSTRACT

A method for improving mitochondrial functions includes a step for supplementing mitochondria with an efficient amount of a reactive persulfide, and a step for increasing a membrane potential of mitochondria by said reactive persulfide.

### CROSS REFERENCE TO RELATED APPLICATIONS

This application is a continuation of U.S. application Ser. No. 17/327,750 filed on May 23, 2021 claiming priorities of U.S. Provisional applications No. 63/029,467 filed on May 23, 2020 under 35 U.S.C. § 119(e), the entire contents of which are hereby incorporated by reference.

### REFERENCE TO SEQUENCE LISTING SUBMITTED VIA EFS-WEB

This application includes an electronically submitted sequence listing XML. The XML file contains a sequence listing entitled “9055_0006PUS2.xml” created on Aug. 27, 2024 and is 13,857 bytes in size. The sequence listing contained in this xml file is part of the specification and is hereby incorporated by reference herein in its entirety.

### BACKGROUND

Early ancestral organisms are believed to have arisen from deep-sea hydrothermal vents where high concentrations of geothermally produced sulfide occur¹. Because sulfide is an important energy source for various microbes that currently live in deep-sea hydrothermal vents and serves as an electron donor for energy production², sulfur-utilizing respiration is likely to be the most primitive type of energy metabolism. Ancient energy metabolism is assumed to utilize hydrogen sulfide—or related sulfur compounds that are yet to be identified—not only as a major substrate but also as an electron acceptor to generate and sustain membrane potential. In contrast, for modern organisms, hydrogen sulfide has been thought to be toxic, particularly for organisms exploiting aerobic energy production, because of its inhibitory effect on the mitochondrial electron transport chain (ETC). However, recent studies have described more positive roles of endogenously produced sulfide as a signaling molecule that regulates angiogenesis^{3,4}, inflammation⁵, autophagy⁶, and aging⁷, although we recently proposed that reactive persulfides may be the molecules that are directly responsible for these functions that are seemingly mediated by hydrogen sulfide^{8,9}.

Enzymes that mediate sulfide oxidation were first described in photosynthetic bacterial¹⁰. Their eukaryotic counterpart, sulfide:quinone oxidoreductase (SQR), is a mitochondrial enzyme and was isolated from a cadmium (Cd)-hypersensitive mutant of a fission yeast¹¹. SQR homologues have been found in worms, fruit flies, mice, and humans^{12,13}, which suggests that sulfide oxidation is widespread among eukaryotes. Indeed, sulfide oxidation occurs in various mammalian cells^{14,15}, and SQR has been thought to play a role in sulfide oxidation mainly as a detoxifier, because excessively high level of cellular SQR activity occurred relative to the rate of endogenous sulfide production that was previously estimated¹⁶. Because all previous studies, as far as we know, used exogenous sulfide to analyze cellular and mitochondrial responses, how endogenously produced sulfide and its oxidation mediated by SQR contribute to mitochondrial sulfur metabolism under physiological conditions remains unknown.

We recently discovered that reactive persulfides such as cysteine hydropersulfide are that reactive persulfides serve as both electron donors and acceptors in the ETC. Our study also demonstrated that impairment of the persulfide-producing activity of CARS2 reduces mitochondrial membrane potential, which indicates that reactive persulfide production in mitochondria is required to maintain membrane potential and possibly efficient production of ATP. That electron acceptance by persulfides is an essential component of metabolic pathways for sulfur-based mitochondrial energy production is therefore highly plausible. Previous studies reported that exogenously supplied sulfide increased oxygen consumption and mitochondrial membrane potential^{17,18}, which implies that, under a particular condition, eukaryotes including mammals may exploit sulfide-based respiration as an evolutionary legacy derived from ancient organisms that lived in sulfide-rich habitats. For this paper, we hypothesized that SQR-mediated mitochondrial energy metabolism.

To prove this hypothesis, we sought to clarify the physiological significance of SQR-mediated hydropersulfide oxidation as a downstream arm of sulfur-based mitochondrial energy production, in which CARS2-mediated persulfide production serves as an upstream arm. We conducted a loss of function study of SQR in fission yeast and mice. For the mouse study, we adopted a unique strategy to eliminate SQR activity from mitochondria by deleting the mitochondrial localization signal at the N terminus of SQR (SQRAN). Analyses of SQR-deficient yeast and SQRAN mice clearly showed that SQR played a fundamental role in mitochondrial energy production and in maintaining the mitochondrial membrane potential for efficient production of ATP. As an important result, the lack of mitochondrial SQR activity in SQRAN mice resulted in growth retardation after weaning and death by 10 weeks after birth, which indicates an essential role of SQR-regulated mitochondrial sulfur metabolism in survival of mice after weaning. Most importantly, hydropersulfides, present as different molecular species including inorganic dihydropersulfide, cysteine hydropersulfide, and glutathione hydropersulfide, served as substrates for SQR and supplied protons in the ETC in mitochondria. Our in vitro and even in vivo studies thus provide robust evidence for the critical requirement of persulfide-mediated mitochondrial energy metabolism, i.e., sulfur respiration, for eukaryotes including mammals under physiological conditions.

### SUMMARY

Sulfur plays a key role in many biological redox reactions. Although sulfur-based respiration is believed to be the most primitive type of energy metabolism, its significance in eukaryotes, especially higher animals, remains to be elucidated. Sulfide:quinone oxidoreductase (SQR), a highly conserved enzyme in various organisms, is thought to catalyze hydrogen sulfide oxidation. Here we found that SQR is essential for persulfide-mediated generation of mitochondrial membrane potential and energy production in yeast and mice. SQR-deficient yeast did not grow on glycerol medium, which demonstrates impaired mitochondrial respiration in the absence of SQR, and longevity of this yeast was markedly attenuated. Mitochondria-selective SQR-deficient mice, which express mutant SQR (SQRAN) lacking a mitochondrial localization signal, died by 10 weeks after birth. Mutant mice manifested compensatory facilitation of β-oxidation, and high-fat diets extended their lifespan. Our precise sulfur metabolome analysis conducted both in vitro and in vivo revealed that SQR effectively catalyzed proton and electron transfer from hydropersulfides, such as cysteine and glutathione hydropersulfides, to the mitochondrial electron transport chain. This SQR-mediated proton supply mediates membrane potential formation leading to energy metabolism and thereby sustains sulfur respiration in mitochondria.

### Results

Sulfur metabolism and impaired mitochondrial energy production in SQR-deficient yeast Eukaryotic SQR was first identified in fission yeast (Schizosaccharomyces pombe) as an enzyme that detoxifies Cd¹¹. To verify the initially described function of SQR, we developed SQR-deficient yeast (Δhmt2) and exposed it to increasing concentrations of Cd. Consistent with results in the previous report¹¹, this SQR-deficient yeast exhibited enhanced susceptibility to Cd toxicity (FIG. 1a). We then examined the SQR contribution to sulfur metabolism. SQR-deficient yeast was more susceptible to high concentrations of sulfide than wild-type (WT) yeast (FIG. 1b). Although, as expected, hydrogen sulfide accumulation was higher in SQR-deficient yeast than in WT yeast, much greater accumulation of cysteine hydropersulfide was observed in cells with deficient SQR expression than in WT yeast (FIG. 1c, Extended Data FIG. 1a). These results thus indicate that cysteine hydropersulfide may be a favorable substrate for SQR besides sulfide in terms of this sulfide metabolism pathway.

To examine the SQR contribution to mitochondrial energy metabolism, we compared yeast growth on different carbon sources-glucose and glycerol (FIG. 1d, Extended Data FIG. 1b). When yeast is cultured in glucose medium, energy production is mediated mainly by glycolysis, independent of mitochondrial respiration. In contrast, energy production during growth in glycerol medium depends fully on mitochondrial energy metabolism¹⁹. Here, yeasts of both genotypes similarly grew well in glucose medium. With glycerol as a sole carbon source, however, growth of SQR-deficient yeasts was limited compared with that of WT yeasts. These results suggest that SQR is a major contributor to mitochondrial energy metabolism.

Of great importance is the result that SQR-deficient yeast did not demonstrate the increase in mitochondrial membrane potential at the stationary phase compared with the log phase that we see in WT yeast (FIG. 1e, Extended Data FIG. 1c), as assessed by 5,5′,6,6′-tetrachloro-1,1′,3,3′-tetraethylbenzimidazolocarbocyanine iodide (JC-1) fluorescence analysis.

This result correlated well with the ATP profile of the WT and SQR-deficient yeasts at each growth phase (FIG. 1f). Notably, the chronological lifespan of SQR-deficient yeast was significantly shorter than that of WT yeast (FIG. 1g). The hydrogen sulfide donor sodium hydrosulfide (NaHS) and hydropersulfide donor sodium disulfide (Na₂S₂) both extended the lifespan of WT yeast but had no effect on SQR-deficient yeast (FIG. 1h). These data suggest that SQR is not merely a Cd-detoxifying enzyme in yeast but truly contributes to bioenergetics for formation of membrane potential in mitochondria, which is likely to be advantageous for lifespan extension in yeast.

### BRIEF DESCRIPTION OF THE DRAWINGS

FIG. 1. shows SQR functions in yeast.

FIG. 2. (SEQ ID NOS: 1-3) shows generation of SQR mutant mice and macroscopic observations of the mice.

FIG. 3. shows metabolic and morphological abnormalities of SQR mutant mice.

FIG. 4. shows energy status of SQR mutant mice.

FIG. 5. shows flux analysis by using ¹³C₁₆-palmitate in SQR mutant MEFs.

FIG. 6. shows sulfur metabolome analysis for SQR mutant mice.

FIG. 7. shows whole-cell evaluation of SQR contribution to mitochondrial activity.

FIG. 8. shows evaluation of the SQR contribution to mitochondrial activity in isolated mitochondria.

### DETAILED DESCRIPTIONS OF THE PREFERRED EMBODIMENT(S)

Mitochondrial SQR Deficiency in Mice Causes Emaciation and Premature Death after Weaning

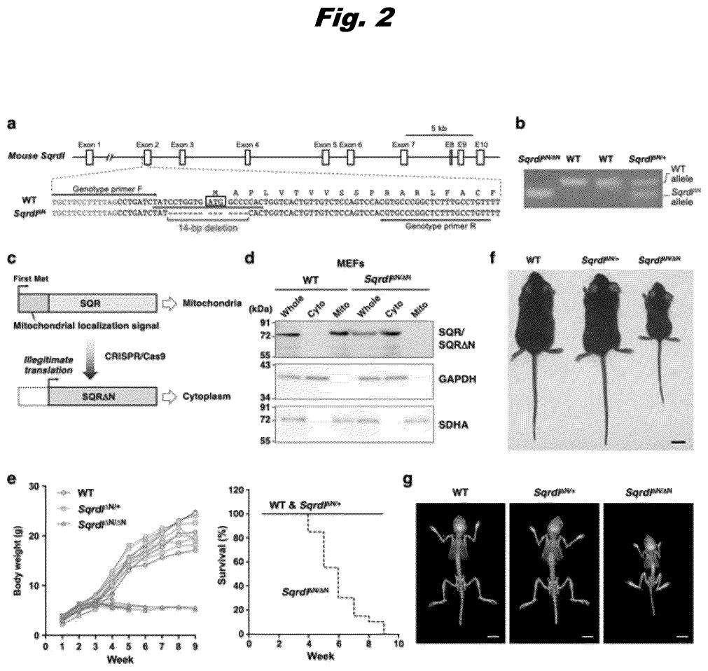

To clarify the mitochondrial function of SQR and reactive persulfide metabolism in higher eukaryotes, we developed mitochondrial SQR-deficient mice that expressed SQRAN, an N-terminally truncated SQR. By using CRISPR-Cas9 technology, we disrupted the translation start codon ATG of the murine Sqrdl gene, which encodes SQR, via a 14-bp deletion (FIG. 2a, b). This change led to an in-frame illegitimate translation from the downstream ATG and resulted in the production of SQRAN lacking the mitochondrial localization signal at the N-terminal region of SQR (FIG. 2c). We investigated the subcellular localization of SQR in mouse embryonic fibroblasts (MEFs) obtained from WT and Sqrdl^{ΔN/ΔN} mice (FIG. 2d).

Mitochondrial localization of SQR that we observed in WT MEFs was totally abrogated in Sqrdl^{ΔN/ΔN} MEFs.

Sqrdl^{ΔN/ΔN} mice were born normally according to the Mendelian ratio (data not shown). Although Sqrdl^{ΔN/ΔN} mice were indistinguishable from WT littermates before weaning, growth of the homozygous mutant animals abruptly ceased at about the weaning period (FIG. 2e, left panel). All Sqrdl^{ΔN/ΔN} mice gradually became emaciated and died within 10 weeks of age, whereas heterozygotes were normal and similar to WT mice (FIG. 2e, right panel). Body size differences between Sqrdl^{ΔN/ΔN} mice and WT controls became apparent after weaning (FIG. 2f, g). Thus, SQR localization in mitochondria is essential for survival of mice after weaning.

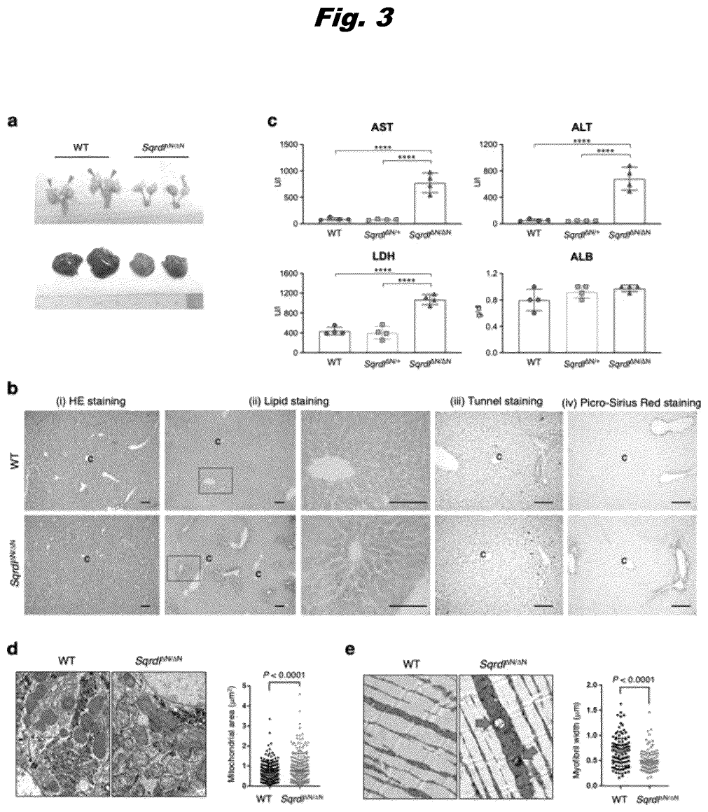

In the peritoneal cavity of Sqrdl^{ΔN/ΔN} mice at 5 weeks after birth, we detected almost no epididymal fat pads, and livers were small and pale (FIG. 3a). Although hematoxylin and eosin (H&E) staining did not reveal clear abnormalities, regions surrounding hepatic triads in Sqrdl^{ΔN/ΔN} mouse livers demonstrated lipid droplet accumulation and fibrotic changes (FIG. 3b). TUNEL staining did not show an apparent increase in apoptotic cells (FIG. 3b), but Sqrdl^{ΔN/ΔN} mice had markedly elevated serum liver enzyme levels (FIG. 3c), which was already apparent at 3 weeks after birth with no obvious blood count abnormalities (Extended Data Table 1). These results suggest that hepatocellular damage is caused primarily by mitochondrial SQR deficiency. As an important result, mitochondrial morphology was also strongly affected by mitochondrial SQR deficiency. Electron microscopy of Sqrdl^{ΔN/ΔN} mouse tissues demonstrated that mitochondria in hepatocytes were oddly shaped and swollen and that mitochondria in skeletal muscle were partially vacuolated (FIG. 3d, e, left panels). Muscle fiber atrophy was also evident in skeletal muscles of mutant mice, compared with WT controls, as mice became emaciated (FIG. 3e, right panel). These mitochondrial abnormalities likely caused the hepatocellular damage, emaciation, and eventual death of Sqrdl^{ΔN/ΔN} mice, which suggests a critical role of SQR in mitochondrial function and sulfur-dependent mitochondrial bioenergetics mediated by SQR.

### Mitochondrial SQR Deficiency Activates β-Oxidation of Fatty Acids

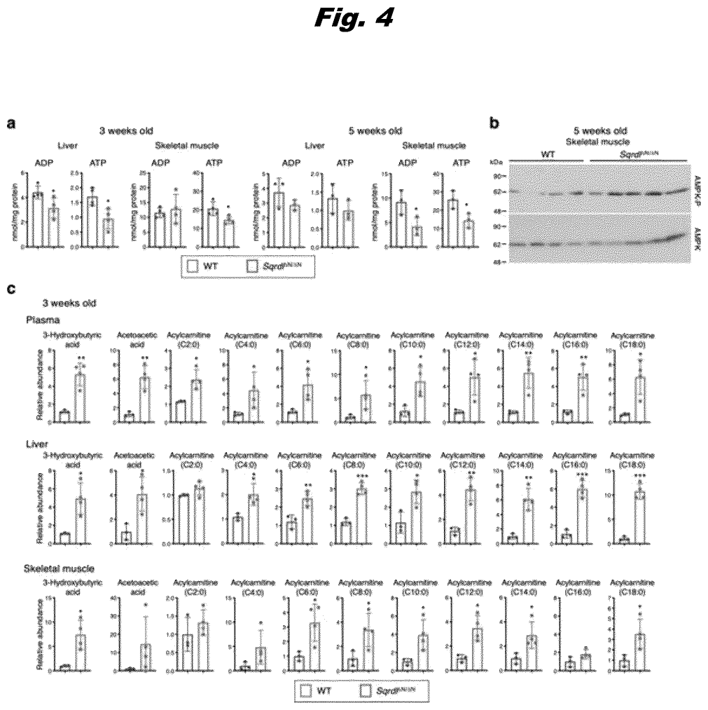

To examine the energy production status related to mitochondrial SQR deficiency, we quantified ATP and ADP levels in livers and skeletal muscles obtained from WT and Sqrdl^{ΔN/ΔN} mice. When mice were 3 weeks old and emaciation of Sqrdl^{ΔN/ΔN} mice was not yet apparent, ATP and ADP levels in livers were reduced in mutant mice (FIG. 4a, left panels). When mice were 5 weeks old, when some Sqrdl^{ΔN/ΔN} mice started dying, ATP and ADP values were reduced, and skeletal muscle of mutant mice manifested AMP-activated protein kinase (AMPK) activation (FIG. 4a, right panels, 4b). Metabolome analysis of plasma, liver, and skeletal muscle of 3-week-old Sqrdl^{ΔN/ΔN} mice commonly showed dramatic increases in acylcarnitine and acyl-CoA species (FIG. 4c, Extended Data FIG. 2). Sqrdl^{ΔN/ΔN} mice also had markedly elevated levels of acetoacetic acid and 3-hydroxybutyric acid (FIG. 4c). These results suggest that β-oxidation of fatty acids is enhanced in the absence of SQR in mitochondria. In addition, Sqrdl^{ΔN/ΔN} mice had elevated levels of branched-chain amino acids (BCAAs) and their catabolites, and glucose was decreased in plasma as well as liver and skeletal muscle (Extended Data FIG. 2), which suggests that catabolism of glucose and BCAAs, as well as that of fatty acids, was promoted.

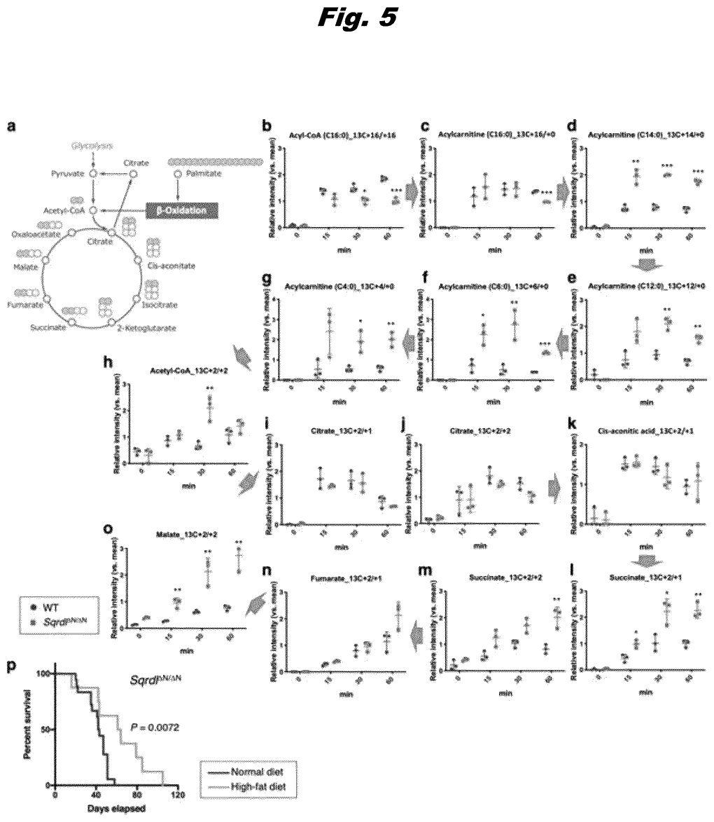

To investigate whether enhanced β-oxidation resulted in increased flux of carbon into the tricarboxylic acid (TCA) cycle, we performed a flux analysis with U-¹³C₁₆-palmitate. Sqrdl^{ΔN/ΔN} and WT MEFs were treated with 150 μM U-¹³C₁₆-palmitate and harvested after 15, 30, and 60 min (FIG. 5a). Fully labeled palmitoyl-CoA (acyl-CoA (C16:0)) and palmitoylcarnitine (acylcarnitine (C16:0)) levels decreased more rapidly in Sqrdl^{ΔN/ΔN} MEFs than in WT MEFs (FIG. 5b, c). Conversely, acylcarnitine species with fully labeled shorter carbon chains and acetyl-CoA increased more rapidly in Sqrdl^{ΔN/ΔN} MEFs than in WT MEFs (FIG. 5d-h). Intermediates carrying two ¹³C atoms increased in the second half of the TCA cycle more rapidly in Sqrdl^{ΔN/ΔN} MEFs than in WT MEFs, which was not apparent in the first half of the TCA cycle (FIG. 5i-o). These results indicate that palmitate consumption is enhanced and that carbons originating from palmitate are utilized in the TCA cycle. Because a high fat Western diet was effective in extending the lifespan of Sqrdl^{ΔN/ΔN} mice (FIG. 5p), enhanced β-oxidation of fatty acids can be regarded as a compensatory mechanism for the energy deficit in the absence of mitochondrial SQR.

### Mitochondrial SQR Deficiency Results in Persulfide Accumulation

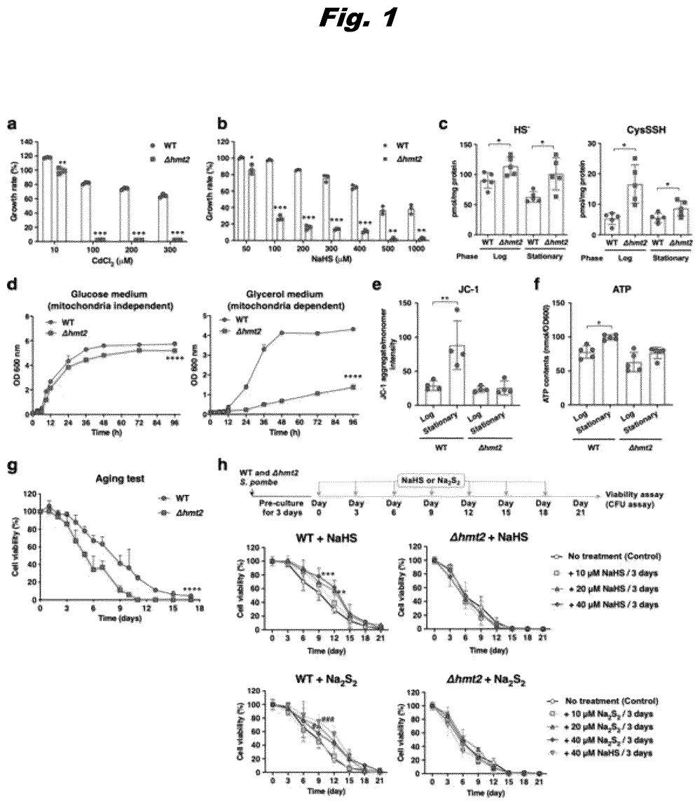

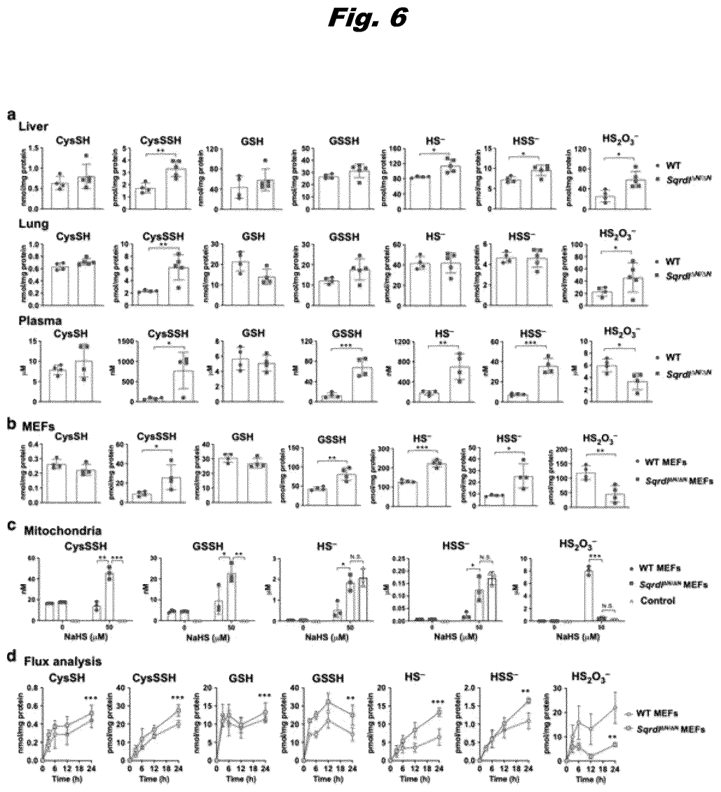

We studied the overall effects of mitochondrial SQR deficiency or of mislocalization of SQR to the cytoplasm on sulfur metabolism in tissues from 3-week-old mice (FIG. 6a, Extended Data FIG. 3a). In plasma of Sqrdl^{ΔN/ΔN} mice, we found a dramatic increase in levels of hydropersulfides, including cysteine hydropersulfide, glutathione hydropersulfide, and inorganic dihydropersulfide in addition to sulfide. In liver and lung, the increase was rather modest but significant for cysteine hydropersulfide. Similarly, Sqrdl^{ΔN/ΔN} MEFs and their isolated mitochondria exhibited elevated levels of hydropersulfides besides hydrosulfide (FIG. 6b, c, Extended Data FIG. 3b, c). These results, together with those observed in yeast (see FIG. 1c), suggest that not only hydrosulfide but also hydropersulfides, such as cysteine hydropersulfide and glutathione hydropersulfide, serve as primary substrates for SQR.

Flux analysis with ³⁴S₂-cystine showed rapid accumulation of cysteine hydropersulfide, glutathione hydropersulfide, dihydropersulfide, and sulfide and reduced production of thiosulfate and oxidized form of glutathione persulfide (i.e., glutathione trisulfide, GSSSG) in Sqrdl^{ΔN/ΔN} MEFs (FIG. 6d, Extended Data FIG. 3d). Inasmuch as thiosulfate is seen as an end product of sulfur respiration mediated by SQR, the flux analysis profile can be interpreted to mean that oxidative catabolism of persulfides and sulfide is impaired by SQR deficiency in mitochondria. The sole accumulation of hydrogen sulfide is not expected to increase hydropersulfides, because the redox equilibrium is normally shifted toward the reduced state and because hydrogen sulfide is too inert to chemically form hydropersulfides through the reaction with simple disulfides such as cystine and GSSG at least under physiological conditions. Therefore, SQR most likely utilized both hydropersulfides and hydrosulfide as substrates.

### Mitochondrial SQR Deficiency Attenuates Mitochondrial Membrane Potential

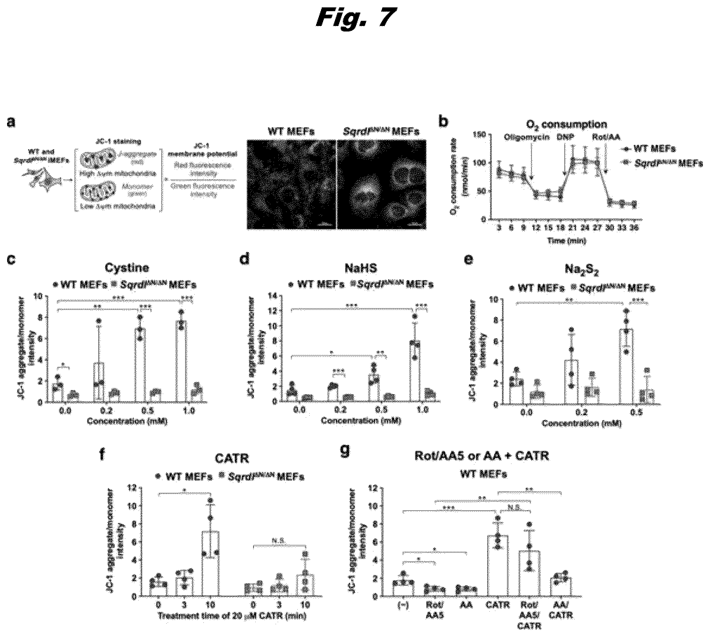

In vivo data strongly suggest that SQR is required for mitochondrial bioenergetics of eukaryotes under physiological conditions. On the basis of our previous studies^{9,20} and other studies^{21,22}, we hypothesized that SQR oxidizes hydrosulfide and hydropersulfide that are generated in mitochondria and contributes to membrane potential formation by transferring electrons and protons to ubiquinone in the ETC. To prove this hypothesis, we investigated the effects of mitochondrial SQR elimination on cellular energy metabolism by using JC-1 fluorescence imaging to assess mitochondrial membrane potential formation. Mitochondrial membrane potential was reduced in Sqrdl^{ΔN/ΔN} MEFs compared with that of WT MEFs under normal culture conditions (FIG. 7a). The oxygen consumption rate (OCR) of Sqrdl^{ΔN/ΔN} MEFs was similar to that of WT MEFs (FIG. 7b), which indicates that electron transport is not significantly affected in Sqrdl^{ΔN/ΔN} MEFs. These results suggest that proton translocation per oxygen consumption is decreased in the absence of mitochondrial SQR. Increasing concentrations of cystine, NaHS, and Na₂S₂ in the culture media of WT MEFs all showed elevation of membrane potential, which was SQR-dependent (FIG. 7c-e). These results suggest that proton/electron donation from hydrosulfide/hydropersulfide to ubiquinone mediated by SQR efficiently generates the membrane potential.

We also analyzed the contribution of SQR to the generation of membrane potential in detail by utilizing specific inhibitors of ETC components (Extended Data FIG. 4). To amplify the membrane potential, we used carboxyatractyloside (CATR), an inhibitor of mitochondrial ADP/ATP translocase²³. CATR treatment increased the membrane potential in WT MEFs, but we did not observe this increase in Sqrdl^{ΔN/ΔN} MEFs (FIG. 7f), which suggests that the proton supply for the proton gradient is reduced in Sqrdl^{ΔN/ΔN} MEFs. The CATR-induced increase in membrane potential in WT MEFs was not significantly affected by rotenone and atpenin A5, inhibitors of complex I and complex II, respectively, but was reduced by antimycin A, an inhibitor of complex III (FIG. 7g). These results suggest that SQR is involved in the proton supply for membrane potential independently of complexes I and II but that it requires complex III activity. Thus, consistent with our hypothesis, SQR likely mediates proton donation from hydrosulfide and hydropersulfide to ubiquinone, which is located downstream of complexes I and II and upstream of complex III.

### Hydrosulfide and Hydropersulfide Generate Mitochondrial Membrane Potential in an SQR-Dependent Manner

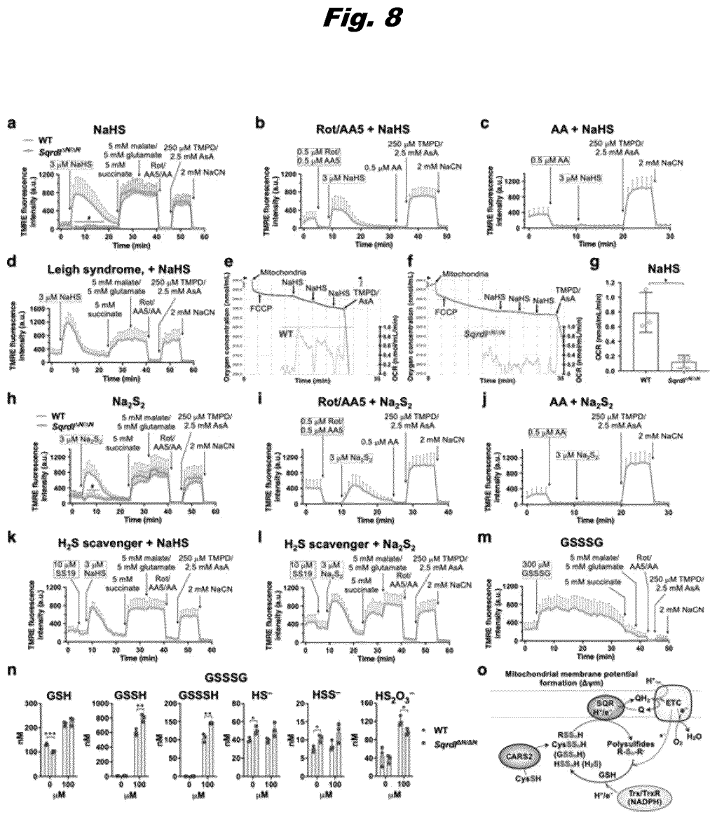

To analyze direct effects of hydrosulfide and hydropersulfide on mitochondrial membrane potential, we established a tetramethylrhodamine ethyl ester (TMRE) time-lapse imaging system of isolated mitochondria. Exploiting this system, we found that sulfide supplementation to isolated mitochondria generated the membrane potential in an SQR-dependent manner, although the effective sulfide dose was much lower for the isolated mitochondria than for the whole cells (FIG. 8a, Extended Data FIG. 5a). Addition of 3 μM NaHS retained the membrane potential response to succinate, malate, and glutamate and to N,N,N′,N′-tetramethyl-1,4-benzenediamine (TMPD) with ascorbate, which indicates that sulfide does not suppress the activities of complex I/II and complex IV. In contrast, the membrane potential response was halted by NaHS of more than 30 μM, which suggests that complex IV was sensitive to sulfide-mediated inhibition (data not shown). The sulfide-mediated generation of membrane potential was not affected by inhibition of complexes I and II (FIG. 8b) but was stopped by complex III inhibition (FIG. 8c). In agreement with these results was the sulfide-generated membrane potential in mitochondria from a patient with Leigh syndrome, which causes a functional impairment of complex I of ETC (FIG. 8d).

To verify electron transport from sulfide, we studied the oxygen consumption of mitochondria isolated from livers of WT and Sqrdl^{ΔN/ΔN} mice. A 10-μM NaHS supply induced a small oxygen consumption only in WT mitochondria (FIG. 8e-g), which indicates that SQR is responsible for the sulfide-derived electron transport. The oxygen consumption induced by the NADH-generating substrates malate and glutamate, followed by a supply of ADP, showed no significant differences between WT and Sqrdl^{ΔN/ΔN} mitochondria (Extended Data FIG. 5b, c), results that are consistent with the whole-cell OCR measured in normal culture media (see FIG. 7b).

Because hydropersulfides may also be substrates for SQR (see FIGS. 1c, 6, and 7), we investigated the effect of hydropersulfides on membrane potential. Addition of Na₂S₂ generated results that were quite similar to those induced by NaHS (FIG. 8h-j), which suggests that the hydropersulfides serves as a comparably favorable substrate for SQR.

Elimination of hydrosulfide by its specific scavenger²⁴, which converts hydrosulfides to hydropersulfides, gave responses that were similar to those of NaHS and Na₂S₂(FIG. 8k, l), which supports the idea that hydropersulfides and hydrosulfide are primary substrates for SQR. The oxidized form of glutathione (GSSSG) persulfide increased the membrane potential (FIG. 8m), which suggests electron acceptance by oxidized persulfides to generate hydropersulfides. Indeed, the addition of GSSSG to isolated mitochondria increased levels of glutathione hydropersulfide (GSSH), which in turn is likely to serve as a substrate for SQR (FIG. 8n). These results demonstrate that a mitochondrial sulfur redox cycle provides persulfides and sulfide that act as electron donors and acceptors and maintains the membrane potential for energy production (FIG. 8o).

### Discussion

This study showed unequivocally an essential function of mitochondrial SQR activity, which suggests that eukaryotes including mammals utilize sulfur respiration under physiological conditions. Oxidation of hydrosulfides and hydropersulfides by SQR, which leads to electron and proton donation to the ETC via ubiquinone and maintenance of the mitochondrial membrane potential, is a missing piece of the sulfur respiration system that we proposed in our previous study^{9,20}. Hydropersulfides produced by CARS2 likely serve as substrates for SQR, as they donate electrons and protons to coenzyme Q (CoQ), and the resulting oxidized persulfides are expected to receive electrons from the ETC and other redox systems in mitochondria (FIG. 8o). The sulfur respiration that is emerging as a new aspect of energy metabolism has updated the concept of cysteinolysis²⁵; cysteine is a source of hydropersulfides, which play a central role not only in cellular redox reactions but also in mitochondrial energy metabolism as they serve as substrates for ATP production.

ETC components in redox reactions have important chemical characteristics: all are electron carriers but only flavin mononucleotide in complex I and CoQ are proton carriers. In particular, CoQ forms a hub entrance for electrons and protons from various metabolites, such as succinate via complex II, acyl-CoA via the electron transfer flavoprotein (ETF) and ETF:ubiquinone oxidoreductase, glycerol-3-phosphate via glycerol-3-phosphate dehydrogenase, and hydrosulfide and hydropersulfide via SQR. That CoQ makes a major contribution to proton transport across the mitochondrial inner membrane is plausible. The emaciation phenotype of Sqrdl^{ΔN/ΔN} mice strongly suggests that protons and electrons supplied via SQR are critical for maintenance of mitochondrial energy homeostasis.

In our previous study, we proposed that cysteine hydropersulfide synthesized by CARS2 served as an electron acceptor from the ETC and that the resulting hydrosulfide served as a substrate for SQR, thus generating the mitochondrial membrane potential⁹. In our current study, however, SQR deficiency in mitochondria unexpectedly caused a large accumulation of hydropersulfides as well as hydrosulfide, which strongly suggests that hydropersulfides are direct substrates for SQR in addition to hydrosulfide. This result allowed us to revise our model of sulfur respiration: cysteine hydropersulfide and derivative hydropersulfides (e.g., GSSH) generated by CARS2 are oxidized by SQR and serve as electron and proton donors in the ETC, and resulting oxidized persulfides (most typically GSSSG) function as electron acceptors in the ETC (FIG. 8o). According to the prevailing viewpoint in the current field of biochemistry, molecular oxygen is believed to be the sole major electron acceptor in the mitochondrial ETC in eukaryotes, but this viewpoint is challenged by the observation that oxidized persulfides are reduced to hydropersulfides in mitochondria (see FIG. 8). Thus, that sulfur respiration in mammals operates under physiological conditions to exploit redox cycles of persulfides is highly plausible. Additional detailed studies are required to obtain solid evidence of persulfides as electron acceptors equivalent to oxygen.

Metabolic flux analysis revealed that a deficiency in mitochondrial SQR activity results in enhancement of fatty acid oxidation. Consistent with the decrease in ATP and ADP in SQR mutant tissues, phosphorylation of AMPK was increased, which likely accounts for enhanced fatty acid oxidation. Because a high-fat diet partially rescued Sqrdl^{ΔN/ΔN} mice, enhanced fatty acid oxidation is thought to be a compensatory response to the energy balance deficit, which unequivocally indicates a critical role of SQR-mediated sulfur metabolism in mitochondrial energy production.

Phenotypic analysis of SQR mutant mice strongly suggests that liver and skeletal muscle, in particular, rely on sulfur respiration for energy production. The sarcopenic phenotype of SQR mutant mice gradually became evident after weaning, when locomotive activity is initiated. Although ATP, which is most actively consumed in contracting skeletal muscles, is believed to be derived mainly from glycolysis and oxidative phosphorylation in white and red fibers, respectively, our results indicate that ATP synthesized from mitochondrial sulfur respiration makes a substantial contribution to maintenance of functional and morphological integrity and metabolic homeostasis of skeletal muscles. In addition, the enhanced fatty acid oxidation may cause continued exacerbation of the sarcopenic phenotype of SQR mutant mice. A recent paper stated that excessive fatty acid oxidation induced oxidative stress and p38 activation in skeletal muscles and consequently muscle atrophy²⁶. Similarly, the liver may have a higher demand for ATP to perform various metabolic and detoxification reactions.

Of note, sulfur respiration does not require a functional complex I, and electrons and protons enter from CoQ and proceed to the ETC, which suggests that dietary supplementation of cystine activates sulfur respiration and increases ATP production in patients with mitochondrial disease, particularly those suffering from complex I insufficiency. A recent paper stated that the Leigh syndrome mouse model, which possesses complex I deficiency due tothe Ndufs4 deletion, is rescued by brain hypoxia²⁷. On the basis of our current study, we hypothesize that hypoxia may drive compensatory induction of sulfur respiration that enables energy production independent of complex I. If this is the case, activation of mitochondrial sulfur metabolism may be a potential therapeutic strategy for mitochondrial diseases with complex I defects as it may stimulate mitochondrial energy metabolism. Promotion of sulfur respiration, besides aiding the treatment of mitochondrial diseases, would benefit the maintenance of healthy tissues and organs with a relatively high demand for energy, such as skeletal muscle, liver, and possibly neural tissues. We thus envision the exciting possibility of an era of novel preventive and therapeutic approaches in various diseases as well as control of aging and longevity.

### Materials and Methods

Construction of the S. pombe HMT2 Deletion Strain

The yeast S. pombe with a 972 background was used in this study. The HMT2-disrupted strain (Δhmt2) was constructed by using a two-step PCR method with minor modifications²⁸. The 5,000-bp homologous sequences were added to the kanMX6 deletion cassette by using a two-step overlapping PCR with the pFA6-kanMX6 plasmid (purchased from the Addgene repository, #39296) and primers (F1: TGAGTTATCGACTGAATATAAACACCTTAT (SEQ ID NO: 4), F2: TTAATTAACCCGGGGATCCGTTTTAGTTGCAAAAATGCAAAAGAGAAGTG (SEQ ID NO: 5), R1: TTAGTAGCATTTTTTCTCTAGATTCTTTTA (SEQ ID NO: 6), and R2: GTTTAAACGAGCTCGAATTCAGATTCAATCATCTTTCTTGCGTAAAATAA (SEQ ID NO: 7)). The PCR fragment was integrated into the genome by transformation via the LiAc/SS carrierDNA/PEG method²⁹. Screening for G418 resistance was performed, and the correct integration event was verified via PCR with chromosomal DNA.

### Growth Inhibition by Cd and NaHS in Yeast

Yeast cells were cultured in synthetic complete medium [SC, 2% glucose, 0.67% yeast nitrogen base without amino acids, and 0.2% dropout supplement (Takara Bio Inc., Shiga, Japan)] at a starting OD₆₀₀ of 0.01. After 10 min at 30° C., CdCl₂ (10-300 μM) or NaHS (50-1,000 μM) was added to the culture, followed by continuing the culture at 30° C. The OD₆₀₀ of the culture was measured after 24 h. Growth rate was converted into percent growth relative to untreated controls.

### Growth Test of Yeast on Glucose and Glycerol Media

For the agar plate culture, yeast cells were grown at 30° C. in YE (3% glucose, 0.5% yeast extract) to the stationary phase and then spotted in 10-fold dilutions at a starting OD₆₀₀ of 1.0 on YE or YEGly medium (3% glycerol, 0.1% glucose, 0.5% yeast extract) containing 2% agar. The plates were then incubated at 30° C. for 2 days. For the liquid culture, yeast cells were cultured at 30° C. in YE or YEGly medium at a starting 0.1 of OD₆₀₀. Cell growth was monitored by measuring OD₆₀₀.

### Measurement of Mitochondrial Membrane Potential in Yeast

The mitochondrial membrane potential was measured by using JC-1 (Molecular Probes, Eugene, OR, USA), a cationic dye that shows potential-dependent accumulation in mitochondria³⁰. Yeast cells were cultured in YE medium at 30° C. for 24 h (log phase) or 48 h (stationary phase), harvested by centrifugation, and washed once with PBS buffer. The harvested cells were incubated with JC-1 (10 μM) in PBS at 30° C. for 30 min and then subjected to flow cytometry analysis. Flow cytometry was performed by using a BD Accuri C6 flow cytometer (BD Biosciences, Franklin Lakes, NJ, USA), and the FL2/FL1 values of individual cells were calculated to determine mitochondrial membrane potential.

### Measurement of ATP in Yeast

Yeast cells were cultured in YE medium at 30° C. for 24 h (log phase) or 48 h (stationary phase) and were then harvested. The harvested cells (about 1×10⁷ cells) were washed three times with ice-cold PBS and suspended in 200 μl of sterilized water. After cells were frozen with liquid nitrogen, they were heated at 95° C. for 10 min to release ATP to the outside of the cells. ATP levels in 100 μl of supernatant were determined by using the IntraCellular ATP assay kit (Toyo B-net, Tokyo, Japan).

### Measurement of Chronological Life Span of Yeast

Three single colonies derived from each strain were analyzed, as previously described³¹ with slight modification. Yeast cells were cultured in SC medium (at a starting OD₆₀₀ of 0.1) at 30° C. with shaking. Measurement of cell viability began after 72 h of culture (day 0) and continued every 2-3 days by plating a fraction of the culture onto a fresh YPD plate (2% glucose, 0.5% yeast extract, 1% peptone, and 2% agar) to determine the number of colony-forming units (CFUs). To investigate the effects of hydrosulfide and hydropersulfide, NaHS (10-40 μM) or Na₂S₂ (10-40 μM) was added every 3 days. Cell survival rates were calculated by normalizing the CFUs at each time point to the CFUs at day 0.

### Generation of Mitochondria-Specific Sqrdl-Deficient Mice

All experimental procedures conformed to the Regulations for Animal Experiments and Related Activities at Tohoku University, were reviewed by the Institutional Laboratory Animal Care and Use Committee of Tohoku University, and were approved by the President of University. We generated Sqrdl-deficient mice as follows. We used B6D2F1 (C57BL/6NCr x DBA/2Cr F1) mice to obtain unfertilized eggs, and we fertilized these eggs in vitro. In vitro fertilization was performed according to a standard protocol with the B6D2F1 strain³². After a 3-h culture of oocytes and sperm, the eggs were removed and cultured for 5 h until electroporation. The Genome Editor electroporator and LF501PT1-10 platinum plate electrode (length: 10 mm, width: 3 mm, height: 0.5 mm, gap: 1 mm) (BEX Co. Ltd., Tokyo, Japan) were used for electroporation. We introduced Cas9/gRNA ribonucleoproteins consisting of the Cas9 protein in complex with a targeting gRNA into fertilized eggs with electroporation, according to protocols previously reported³³, after which we transferred the eggs to oviducts of pseudo-pregnant females on the day of the vaginal plug. The sequence of the gRNA was designed as follow: 5′-TATCCTGGTGATGGCCCCAC-3′ (SEQ ID NO:8), located at exon 2 of the Sqrdl gene to generate Sqrdl-mutant mice. A founder mouse harboring the Sqrdl mutant allele with a 14-bp deletion spanning the translation initiation codon in exon 2 (Sqrdl^{ΔN}) was crossed with WT mice to obtain Sqrdl heterozygous (Sqrdl^{ΔN/+}) mice. Sqrdl^{ΔN/+} mice were back-crossed to the C57BL/6j background for more than six generations. Genotyping was performed by means of PCR and gel electrophoresis of the PCR product. The genotyping primers were 5′-TGCTTCCTTTTAGCCTGATCTA-3′ (SEQ ID NO: 9) and 5′-AAAACAGGCAAAGAGCCGGGCAC-3 (SEQ ID NO: 10).

### Establishment of Immortalized MEFs

WT and Sqrdl^{ΔN/ΔN} immortalized MEFs (iMEFs) were established from mouse embryos at E13.5 and immortalized by lentiviral introduction of SV40 large T antigen. WT and Sqrdl^{ΔN/ΔN} embryos were obtained from Sqrdl^{ΔN/+} pregnant females mated with Sqrdl^{ΔN/+} males. Stable transformants were selected via 2 μg/ml puromycin, and three independent WT iMEF lines and Sqrdl^{ΔN/ΔN} iMEF lines were established.

### Localization of SQR by Western Blotting

Mitochondrial fractions were isolated from WT and mitochondria-specific Sqrdl-deficient iMEFs as previously described³⁴. Briefly, cells were homogenized in isotonic buffer (10 mM HEPES, pH 7.4, 75 mM sucrose, 225 mM mannitol, and 2 mM EDTA) with a Teflon homogenizer for 50 strokes at 1,600 rpm and centrifuged at 700 g at 4° C. for 10 min. The supernatants were centrifuged again at 5,000 g at 4° C. for 10 min. The pellets were washed twice with the isotonic buffer and used as mitochondrial fractions. The soluble fractions obtained by this process were filtered through a 0.22-mm filter and used as cytosolic fractions. Western blot analysis was performed by using anti-SQR, anti-GAPDH (Santa Cruz Biotechnology, Santa Cruz, CA, USA; clone: sc-25778), and anti-SDHA (succinate dehydrogenase subunit A) (Abcam, Cambridge, UK; clone: ab14715). The polyclonal antibody for mouse SQR was produced by immunizing rats with recombinant mouse SQR. Samples were solubilized with Laemmli lysis buffer (125 mM Tris HCl, 2% SDS, 20% glycerol, and 10% 2-mercaptoethanol, pH 6.8), loaded on SDS-PAGE, and transferred to polyvinylidene fluoride membranes (GE Healthcare, Little Chalfont, England). The membranes were blocked with Blocking One (Nacalai Tesque, Kyoto, Japan) at room temperature for 60 min and were incubated with primary antibodies (1/5,000) diluted by Can Get Signal Immunoreaction Enhancer Solution 1 (TOYOBO, Osaka, Japan) at 4° C. overnight. After membranes were washed with TBST buffer (50 mM Tris-HCl, 150 mM NaCl, 0.1% Tween 20, pH 7.4), they were incubated with horseradish peroxidase-conjugated secondary antibody (1/5,000) diluted by Can Get Signal Immunoreaction Enhancer Solution 2 (TOYOBO) at room temperature for 1 h. Immunoreactive bands were detected by using an ECL Prime Western Blotting Detection Reagent (GE Healthcare) with a luminescent image analyzer (ImageQuant LAS 500; GE Healthcare).

Analysis of blood counts and blood biochemical profiles Blood was drawn from the posterior vena cava. Hematological indices were measured with an automatic blood cell analyzer (Nihon Kohden, Tokyo, Japan). Plasma was analyzed by using Fuji Dri-Chem 7000 (Fujifilm Corp., Tokyo, Japan) to detect aspartate aminotransferase (AST), alanine aminotransferase (ALT), lactate dehydrogenase (LDH), and albumin (ALB).

### Histological Analysis

Mouse tissues were fixed overnight in Mildform ION (Fujifilm Wako Pure Chemical Corp., Osaka, Japan) at 4° C. Lipids in frozen liver sections were stained with oil red O solution (Muto Pure Chemicals, Tokyo, Japan) as described elsewhere. For other histological analyses, fixed tissues were dehydrated in alcohol and embedded in paraffin, and 6-μm serial sections were prepared. H&E staining was performed according to standard procedures. Picrosirius red staining for collagens and the TUNEL assay for detecting cell death were performed with the Picro-Sirius Red Stain Kit (Polysciences Inc., Warrington, PA, USA) and In situ Apoptosis Detection Kit (Takara, Ohtsu, Japan), respectively. Slides used for the TUNEL assay were counterstained with methyl green (Sigma-Aldrich, St. Louis, MO, USA).

### Computed Tomography (CT) Analyses

Mice were anesthetized with isoflurane and scanned by using a Latheta (LCT-200) experimental animal CT system (Hitachi-Aloka Medical, Tokyo, Japan). Continuous slice images of the abdominal areas from L1 to the sacral division were obtained. The Latheta software was used to determine fat and lean areas on the abdominal CT images as measured in Hounsfield units (HU).

### Transmission Electron Microscopy for Assessment of Mitochondrial Morphology

To investigate morphology of mitochondria from WT and SqrdlDN/DN mice, we performed transmission electron microscopy, as previously described³⁵. Tissues were fixed with 2% glutaraldehyde/2% paraformaldehyde in 0.15 M cacodylate buffer on ice for 2 h, followed by examination with an electron microscope.

### Measurement of Metabolites in Mouse Tissue

### Quantification of ATP and ADP

For quantification of ATP and ADP, mice were killed by cervical translocation, and liver and skeletal muscle were immediately dissected and snap-frozen in liquid nitrogen. The frozen tissues were homogenized with ice-cold 10% TCA with a Psychotron homogenizer (Microtec Co. Ltd., Chiba, Japan). After centrifuation at 4° C. for 10 min at 15,000 rpm, the supernatants were diluted with stable isotope-labeled ¹³C₁₀-ATP (Sigma-Aldrich) and measured by using liquid chromatography-electrospray ionization tandem mass spectrometry (LC-ESI-MS/MS) (Shimadzu, Kyoto, Japan; LCMS-8060). The triple quadrupole (Q) mass spectrometer LCMS-8060 coupled with the Nexera UHPLC system (Shimadzu) was used to perform LC-ESI-MS/MS. ATP and ADP were separated by means of Nexera UHPLC with a Mastro C18 column (Shimadzu; 100 mmL×2.1 mm, I.D., 3 m) under the following elution conditions: mobile phase A (10 mM NH₄HCO₃/acetonitrile, 90/10) with a linear gradient of mobile phase B (50 mM NH₄HCO₃/acetonitrile, 70/30) from 0% to 100% for 20 min at a flow rate of 0.3 ml/min at 40° C. ATP and ADP were identified and quantified by means of multiple reaction monitoring (MRM). The negative mode MRM parameters of ATP and ADP were ATP, 506>159, collision energy (CE) 29 V; ¹³C₁₀-ATP, 516>159, CE 29 V; and ADP, 426>134, CE 22 V. Quantification of ADP was determined from the value of ¹³C₁₀-ATP by calculating the ionization efficiency of ADP from the standard ATP/ADP ratio. Tissue pellets were reconstituted in 0.1% SDS in PBS buffer, homogenized by sonication, and centrifuged again at 15,000 rpm at 4° C. for 10 min. The supernatant was used for the bicinchoninic acid (BCA) protein assay to determine the total protein content of the samples.

### Sample Preparation for Metabolome Analysis

Plasma, liver, and skeletal muscles were prepared as follows. Venous blood was drawn from anesthetized mice by using a dry sterile plastic syringe and was immediately transferred to EDTA-treated plastic tubes. Plasma was obtained by centrifugation at 3,000 rpm at 4° C. for 2 min. After blood was drawn, the mice were killed, and liver and skeletal muscles were snap-frozen in liquid nitrogen.

### Metabolome Analysis Using Mass Spectrometry

Metabolome profiles were obtained by combining data derived from two analytical platforms: liquid chromatography-tandem mass spectrometry (LC-MS/MS) and gas chromatography-tandem mass spectrometry (GC-MS/MS). Tissues were homogenized in methanol (100 mg/ml) via a ShakeMaster Auto (Biomedical Science Inc., Tokyo, Japan), and samples were centrifuged at 15,000 rpm for 5 min. Supernatants were applied to each analysis.

For LC-MS/MS analysis, 100 μl of supernatant was mixed with 25 μl of 100 mM ammonium formate. The mixture was vortexed and centrifuged at 15,000 rpm for 5 min. Then 2 μl of supernatant was injected into a LC-MS/MS system consisting of the UHPLC Nexera LC (Shimadzu) and a 5500 QTRAP mass spectrometer (AB Sciex Pte. Ltd., Toronto, Canada). Chromatographic separation was performed via the ZIC-cHILIC column (2.1×100 mm, 3 μm; Merck Millipore, Darmstadt, Germany), with the column temperature at 30° C., by gradient elution of mobile phase A (10 mM ammonium formate aqueous solution) and mobile phase B (acetonitrile). The gradient program was as follows: 0-1.5 min, 97% B; 1.5-5 min, 97-75% B; 5-7 min, 75% B; 7-10 min, 75-40% B; 10-12 min, 40% B; 12-13 min, 40-10% B; 13-16 min, 10% B; and 16-25 min, 97% B, with a flow rate of 0.4 ml/min. The LC eluate was directly introduced into a turbo spray ionization source with simultaneous polarity switching, whose ionization parameters appear in Extended Data Table 2. MRM was used to detect metabolites, with the detection conditions set on the basis of information in a previous report³⁶. LC-MS/MS data were processed via MultiQuant 3.0 (AB Sciex Pte. Ltd.).

For GC-MS/MS analysis, 10 μl of the homogenized supernatant was dried by using nitrogen stream, after which it was processed by two-step reactions: oximation and trimethylsilylation. Oximation was achieved by adding 25 μl of O-methylhydroxylammonium chloride in pyrimidine (15 mg/ml) and incubating the sample at 40° C. for 60 min. Then, for trimethylsilylation, 25 μl of N, O-bis(trimethylsilyl)trifluoroacetamide with 1% trimethylchlorosilane was added to the reaction solution, and incubation proceeded at 60° C. for 60 min. The reaction mixture, 1 μl, was injected into an Agilent 7890B series GC system in the split injection mode (10:1, v/v) via a GC injector 80 autosampler (Agilent Technologies Inc., Santa Clara, CA, USA). GC separation was performed with a J&W Scientific DB-5MS-DG column (30 m×0.25 mm i.d., df=0.25 m; Agilent Technologies Inc.) with a temperature gradient, which rises at 10° C./min from 60° C. to 325° C., and with a constant helium gas flow at 1 ml/min. The elution was ionized by means of electron impact ionization (70 eV) with an ion source temperature of 280° C., and it was introduced into an Agilent 7010B triple-quadrupole mass spectrometer. Each target molecule was detected by MRM and its peak area was calculated by using MassHunter software (Agilent Technologies Inc.).

### Immunoblot Analysis of Mouse Skeletal Muscle

The intermediate portions of the gastrocnemius were dissected free immediately after cervical dislocation of the mice, after which the samples were sonicated in 2× loading buffer (100 mM Tris-HCl, pH 6.8, 4% SDS, 10% glycerol, and 2 mM dithiothreitol). The whole-cell lysates were separated via 10% SDS-polyacrylamide gel. The antibodies AMPKα (D5A2; Cell Signaling Technology, Danvers, MA, USA; 1:1,000) and phospho-AMPKα (Thr172) (40H9; Cell Signaling Technology; 1:1,000) were used. Chemi-Lumi One L (Nacalai Tesque) was used for detection.

Flux Analysis with ¹³C₁₆-Palmitate
Preparation of ¹³C₁₆-Palmitate Solution

¹³C₁₆-palmitate powder (cat #605573; Sigma-Aldrich) was dissolved in preheated 0.1 M NaOH and diluted 1:10 in a prewarmed solution of 5% sterile bovine serum albumin (cat #01863-77; Nacalai Tesque) to give a final stock concentration of 5 mM. The ¹³C₁₆-palmitate solution was filter-sterilized (0.2 m) and stored at −20° C. The solution was preheated at 70° C. for 30 min before use. Treatment of MEFs with ¹³C₁₆-palmitate

MEFs—8×10⁵ WT and 5×10⁵ Sqrdl^{ΔN/ΔN} MEFs—were seeded in DMEM (cat #044-29765; Wako, Japan) supplemented with 10% fetal bovine serum (FBS) and penicillin-streptomycin and were cultured at 37° C. in humidified air-5% CO₂ for 24 h. Triplicate samples were prepared for each genotype. The medium was changed to prewarmed glucose-deficient DMEM (cat #042-32255; Wako, Japan) supplemented with 10% FBS and penicillin-streptomycin and incubated for another 3 h. The medium was changed to the same prewarmed medium containing ¹³C₁₆-palmitate at a final concentration of 150 μM. Then, 15, 30, and 60 min after addition of ¹³C₁₆-palmitate, cells were harvested in 1 ml of methanol and snap-frozen in liquid nitrogen.

Measurement of ¹³C₁₆-Labeled Metabolites

Samples were evaporated to dryness under a steady stream of nitrogen at 40° C. The residual was reconstituted with 90% MeOH (200 μl/1 e6 cells). Cell suspensions were vortexed and centrifuged at 15,000 rpm for 5 min. The 5-μl of supernatant was injected into a LC/MS/MS system consisting of a UHPLC Nexera liquid chromatography system (Shimadzu co., Kyoto, Japan) and a 5500QTRAP mass spectrometer (AB Sciex pte. ltd., Toronto, Canada) equipped with a Luna C18(2) column (2×100 mm, 3 m, 100 Å; Phenomenex Inc., Torrance, CA, USA) and was maintained at 40° C.; the metabolites were separated by gradient elution of mobile phase A-0.1% octylamine, 0.07% acetic acid, and 10 μM EDTA-2Na in MilliQ water—and of mobile phase B—0.07% acetic acid in acetonitrile. The gradient was as follows: 1% B for 2 min; a linear increase from 1% to 100% B in 8 min; and 100% B for 5 min. All target molecules were observed via MRM; MRM conditions appear in Extended Data Table 3. MS/MS data were processed by using MultiQuant 3.0 (AB Sciex Pte. Ltd.).

### Western Diet Treatment

Sqrdl^{ΔN/ΔN} mice at about 10 days after birth, which is before weaning, and their mothers were fed the Western diet D12079B (Research Diet, Inc., New Brunswick, NJ, USA) or normal diet. Survival of these two groups of mice was studied.

### Measurement of MEF Membrane Potential

To determine the membrane potential of mitochondria under several experimental conditions, JC-1 staining was performed according to the method previously reported 37,38. Accumulation of the cell-permeable JC-1 probe (Abcam) in mitochondria depends on the membrane potential and is associated with a fluorescence emission shift from green (emission 527 nm) to red (emission 590 nm). Briefly, WT and Sqrdl^{ΔN/ΔN} MEFs, cultured in 8-well multichamber slides (Matsunami, Osaka, Japan) coated with collagen, were treated with mitochondrial ETC inhibitors (rotenone, atpenin A5, and antimycin A) the and ADP/ATP carrier inhibitor CATR²³ as described in the figure legends. For JC-1 staining, cultured cells were washed with HKRB buffer (20 mM HEPES, 103 mM NaCl, 4.77 mM KCl, 0.5 mM CaCl₂), 1.2 mM MgCl₂, 1.2 mM KH₂PO₄, 25 mM NaHCO₃, and 15 mM glucose, pH 7.3), incubated with 20 M JC-1 at 37° C. for 20 min, rinsed twice with HKRB buffer, and examined with a confocal laser scanning microscope (Nikon C2 plus, with NIS-Elements 5.01 software). ImageJ software was used for image processing and calculation of JC-1 fluorescence intensities.

Measurement of the OCR of WT and Mitochondria-Specific Sqrdl-Deficient iMEFs

The OCR of mitochondria from WT and mitochondria-specific Sqrdl-deficient iMEFs was measured by using the XF96 Extracellular Flux Analyzer (Agilent). The OCR was determined under various conditions (oligomycin, dinitrophenol, and rotenone plus antimycin A). Using these metabolic modulators allows multiple parameters of mitochondrial function to be determined, as previously reported³⁹. Oligomycin was used to inhibit ATP synthase, 2,4-dinitrophenol (DNP) to uncouple mitochondria and yield maximal OCR, and rotenone plus antimycin A to inhibit mitochondrial oxygen consumption.

### Measurement of the Membrane Potential of Isolated Mitochondria

### Preparation of MEF Mitochondria

MEF mitochondria were isolated by differential centrifugation. WT and Sqrdl^{ΔN/ΔN} MEFs were homogenized in the extraction buffer (10 mM Tris-HCl, 250 mM sucrose, and 0.5 mM EGTA, pH 7.4) with a Dounce homogenizer (Teflon glass) and were centrifuged at 2,000 g at 4° C. for 5 min to remove nuclei and cell debris. The supernatants were centrifuged at 12,000 g at 4° C. for 10 min, and mitochondrial pellets were washed three times with buffer and kept at −80° C. until use.

### Time-Lapse Imaging of Membrane Potential in Single Mitochondria

To determine the membrane potential in isolated mitochondria under several experimental conditions, TMRE staining was performed according to the method previously reported⁴⁰. The TMRE fluorescence intensity at a maximum emission of 574 nm depends on the mitochondrial membrane potential. Briefly, mitochondria from WT and Sqrdl^{ΔN/ΔN} MEFs were adsorbed on the bottom of 8-well μ-Slides (Ibidi, Martinsreid, Germany) coated with poly-D-lysine (Sigma-Aldrich, St. Luis, MO, USA), after which they were stained with 10 nM TMRE (Biotium, Fremont, CA, USA) and 50 nM MitoTracker Green FM (Thermo Fisher Scientific, Waltham, MA, USA) at 25° C. for 10 min. TMRE fluorescence intensity (561 nm excitation and 605±15 nm emission), which is positive with MitoTracker Green FM fluorescence, was measured in individual mitochondria by using the region of interest (ROI) tool of the confocal laser scanning microscope (Nikon C2 plus, with NIS elements version 5.01 software). ROIs, 200 each for WT and Sqrdl^{ΔN/ΔN} mitochondria, were manually drawn around a single mitochondrion that did not overlap with others, and the average non-zero pixel intensity within the ROIs was measured and plotted against time as arbitrary units. Because damage to some mitochondria is technically inevitable, we selected mitochondria that showed a sufficient response to TMPD/ascorbate. Specifically, of 200 ROIs, the top 30 ROIs were selected for analysis of TMRE fluorescence intensity in response to TMPD/ascorbate, and their means and s.d. values were calculated.

### Measurement of the OCR of Liver Mitochondria

### Isolation of Liver Mitochondria

Mitochondria were isolated from 4- to 6-week-old mice by using two-step differential centrifugations, as previously described⁴¹ with minor modifications. In brief, after gallbladder removal, fresh liver specimens weighing 1.0-1.5 g were minced and homogenized in ice-cold isolation buffer (10 mM Tris/MOPS, 1 mM EGTA/Tris, and 200 mM sucrose, pH 7.4). When liver weight was less than 1 g, livers were pooled. Homogenate were centrifuged at 600 g and 4° C. for 10 min to remove cellular debris and nuclei. Supernatants containing mitochondrial fraction were then centrifuged at 7,000 g at 4° C. for 10 min. Obtained mitochondrial pellets were washed with new isolation buffer and underwent centrifugation again (7,000 g for 10 min). Supernatants were discarded, and the final mitochondrial pellet was resuspended in a remaining minimal volume of the isolation buffer. The protein concentration was determined with the BCA Protein Assay Kit (Thermo Fisher Scientific). Absorbance at 562 nm was measured with NanoDrop 2000 (Thermo Fisher Scientific).

### Measurement of Liver Mitochondrial Respiratory Function

Mitochondrial oxygen consumption was measured by using a Clark-type electrode (Oxytherm system; Hansatech Instruments, Norfolk, UK). All experiments were conducted with isolated mitochondria, 1 mg protein/ml in experimental buffer (125 mM KCl, 10 mM Tris/MOPS, 10 mM EGTA/Tris, and 1 mM Pi, pH 7.4), with continuous stirring at 25° C. The OCR through complex I was measured as a mitochondrial basic function by sequentially adding 2.5 mM malate/5 mM glutamate, 150 μM ADP, and 100 nM carbonyl cyanide 4-(trifluoromethoxy) phenylhydrazone (FCCP). The respiratory control index (RCI) was calculated by using the ratio of state 3 to state 4. The electron donation ability of SQR was analyzed by three additions of 10 μM NaHS in the presence of 100 nM FCCP, which was followed by addition of 250 μM TMPD/2.5 mM ascorbic acid for evaluation of complex IV integrity. Then, 100 μM sodium cyanide (NaCN) was added to terminate the electron transfer.

### Quantification of RSS

Yeast, mouse tissues, and MEFs were analyzed for the low-molecular-weight analytes cysteine (CysSH), cysteine hydropersulfide (CysSSH), glutathione (GSH), glutathione hydropersulfide (GSSH), bis-S(H₂S), bis-SS (HSSH), cystine (CysSSCys), oxidized glutathione, orglutathione disulfide (GSSG), oxidized glutathione trisulfide (GSSSG), and thiosulfate (HS₂O₃) as previously described⁹. Briefly, samples were homogenized with a Psychotron homogenizer (Microtec Co. Ltd.) or sonicator in ice-cold methanol in the presence of 5 mM A-(4-hydroxyphenyl)ethyl iodoacetamide (HPE-IAM). Homogenates were incubated at 37° C. for 20 min and then centrifuged at 15,000 rpm at 4° C. for 10 min. Supernatants were separated and diluted 10 times with 0.1% formic acid. Stable isotope-labeled internal standards dissolved in 0.1% formic acid solution were added to the samples to a final concentration of 25 nM. A sample of 10 μl was injected into the LC-ESI-MS/MS instrument (LCMS-8060). Various persulfide derivatives were identified and quantified by means of MRM, as previously described⁹. Cell pellets were reconstituted in 0.10% SDS in PBS buffer, homogenized by sonication, and centrifuged again at 15,000 rpm at 4° C. for 10 min. The BCA protein assay was performed with the supernatants to determine the total protein content of the samples. Each analyte concentration was normalized to the measured protein concentration. Isolated mitochondria (0.45 mg/ml) from WT and Sqrdl^{ΔN/ΔN} MEFs were treated with 50 μM NaHS and 100 μM GSSSG, or were not treated, at 37° C. for 60 min and 15 min, respectively. After incubation, RSS were extracted with 5 mM HPE-IAM containing methanol and subjected to LC-ESI-MS/MS analysis by following the procedure described above.

### Sulfur Flux Analysis

³⁴S-labeled cysteine was synthesized from ³⁴S-labeled NaHS and L-serine by using recombinant cysteine synthases CysK, as previously described⁴². Synthesized ³⁴S-labeled cysteine was oxidized by H₂O₂ to form ³⁴S₂-cystine. The synthesized ³⁴S₂-cystine was purified via high-performance LC. For quantification of cellular ³⁴S-labeled flux analysis, novel stable isotope-labeled internal standards were prepared by using [d4]-labeled HPE-IAM (Toronto Research Chemicals, North York, Ontario, Canada), as previously described⁹. WT and Sqrdl^{ΔN/ΔN} MEFs were treated with 0.2 nM ³⁴S₂-cystine in normal cystine-free DMEM (10% FBS and 1% penicillin and streptomycin solution) for 24 h at 37° C. After incubation, cells were washed with PBS and extracted with 5 mM HPE-IAM containing methanol followed by the procedures described above. Samples containing 25 nM stable isotope-labeled internal standards were subjected to LC-ESI-MS/MS (LCMS-8060). Various per/polysulfide derivatives were identified and quantified by means of MRM. Extended Data Table 4 summarizes the MRM parameters for ³⁴S flux analysis.

### Statistical Analysis

Statistical significance was evaluated by using an unpaired two-sample Student's t-test for two-group comparisons and one-way/two-way ANOVA with Tukey's test for multiple-group comparisons. Kaplan-Meier analysis was used for survival of Sqrdl^{ΔN/ΔN} mice, and statistical significance was evaluated via the log-rank test. These analyses were performed by using Microsoft Office Excel (Microsoft) and Prism 7 (GraphPad Software, Inc., San Diego, CA, USA). P<0.05 was considered to be statistically significant.

### DISCUSSIONS ON THE DRAWINGS

This section adds discussions on the drawings (FIGS. 1 to 8).

### FIG. 1. SQR Functions in Yeast.

a, Effect of CdCl₂ treatment on yeast growth. Results are presented as means±s.d. of three independent experiments, with statistical significance determined via Student's t-test. **P<0.01, ***P<0.001, Δhmt2 vs. WT. b, Effect of NaHS treatment on yeast growth. Results are presented as means±s.d. of three independent experiments, with statistical significance determined by Student's t-test. *P<0.05, **P<0.01, ***P<0.001, Δhmt2 vs. WT. c, Profiles of sulfur metabolism during growth in glycerol media. Results are presented as means±s.d. of five independent experiments, with statistical significance determined by Student's t-test. *P<0.05, Δhmt2 vs. WT. d, Yeast growth in glucose medium (left panel) and glycerol medium (right panel). Data are presented as means±s.d. of three independent experiments, with statistical significance determined by two-way ANOVA with Tukey's test. ****P<0.0001, Δhmt2 vs. WT. e, Mitochondrial membrane potential evaluation by JC-1 at log and stationary phases. Average fluorescence ratios, JC-1 aggregates (red) vs. JC-1 monomers (green), are shown (see Extended Data FIG. 1c). Data are presented as means±s.d. of four independent experiments. **P<0.01, Δhmt2 vs. WT, determined by one-way ANOVA with Tukey's test. f, ATP levels at log and stationary phases. Data are presented as means±s.d. of five independent experiments. *P<0.05, Δhmt2 vs. WT, determined by one-way ANOVA with Tukey's test. g, Chronological survival curves. Values are presented as means±s.d. of five independent experiments. ****P<0.0001, Δhmt2 vs. WT, determined by two-way ANOVA with Tukey's test. h, Chronological survival curves of yeasts supplemented with NaHS or Na₂S₂. Values are presented as means±s.d. of five independent experiments. **P<0.01 and ***P<0.001 for 20 μM and 40 μM NaHS treatment vs. control, respectively. #P<0.05 and ###P<0.001 for 20 μM Na₂S₂ and 40 μM NaHS treatment vs. control, respectively. Significance was determined via two-way ANOVA with Tukey's test.

### FIG. 2. Generation of SQR Mutant Mice and Macroscopic Observations of the Mice.

a, Schematic illustration of the murine Sqrdl gene structure and sequences of WT and mutant alleles spanning the translation initiation codon. Blue letters and black letters indicate the first intron and the second exon, respectively. ATG enclosed by a box indicates the first methionine codon. A target sequence of gRNA is underlined. Target sequences for genotyping primers are indicated by arrows. b, PCR detection of a deletion in the Sqrdl gene. Genomic DNAs from WT, Sqrdl^{ΔN/+}, and Sqrdl^{ΔN/ΔN} mice were amplified with a primer set shown in panel a. c, Schematic presentation of SQR proteins expressed by WT and Sqrdl^{ΔN} alleles. d, Immunoblot analysis of whole-cell lysates (Whole) and cytosolic (Cyto) and mitochondrial (Mito) fractions of MEFs established from WT and Sqrdl^{ΔN/ΔN} embryos. Protein samples were prepared for detection of SQR, GAPDH (cytosolic marker), and SDHA (mitochondrial marker). e, Body weight gain (left panel) and survival (right panel) of Sqrdl^{ΔN/ΔN} mice (n=8) compared with Sqrdl^{ΔN/+} mice (n=6) and WT mice (n=3). f, Macroscopic appearance of 5-week-old WT, Sqrdl^{ΔN/+}, and Sqrdl^{ΔN/ΔN} littermate male mice. Scale bar, 1 cm. g, Microtomography photographs of the whole bodies of 5-week-old WT, Sqrdl^{ΔN/+}, and Sqrdl^{ΔN/ΔN} littermate male mice. Scale bars, 1 cm. Representative images of more than three of each genotype are shown (f,g).

### FIG. 3. Metabolic and Morphological Abnormalities of SQR Mutant Mice.

a, Representative macroscopic images of epididymal fat pads (blue arrowheads in the upper panel) and livers (bottom panel) from 5-week-old WT and Sqrdl^{ΔN/ΔN} male mice (more than 10 each). b, Representative histological micrographs of liver sections from 5-week-old WT and Sqrdl^{ΔN/ΔN} male mice (n=3). Scale bars, 100 μm. Right panels in (ii) are higher magnifications of areas outlined by rectangles in left panels in (ii). c, central vein. c, Serum biochemical test that measured markers of liver injury (AST, ALT, and LDH) and ALB in 3-week-old mice (n=4). ****P<0.0001, determined by one-way ANOVA with Tukey's test. d,e, Electron micrographs of liver (d) and skeletal muscle (e) of 3-week-old WT and Sqrdl^{ΔN/ΔN} mice. The mitochondrial area (d) and myofibril width (e) were measured. Red arrows indicate vacuolated mitochondria. Representative data are shown for three mice of each genotype.

### FIG. 4. Energy Status of SQR Mutant Mice.

a, ADP and ATP quantification in livers and skeletal muscles of 3-week-old (n=4) and 5-week-old (n=3) WT and Sqrdl^{ΔN/ΔN} mice. b, Immunoblot analysis of whole-cell lysates prepared from skeletal muscles of 5-week-old WT (n=4) and Sqrdl^{ΔN/ΔN} (n=5) mice. AMPK and its phosphorylated form were detected. c, Relative quantities of β-oxidation intermediates in plasma, livers, and skeletal muscles of 3-week-old mice. Data are presented as means±s.d. (WT n=3; Sqrdl^{ΔN/ΔN} n=4). *P<0.05, **P<0.01, ***P<0.001, determined by Student's t-test.

FIG. 5. Flux Analysis by Using i³C₁₆-Palmitate in SQR Mutant MEFs.

a, Metabolic pathway of β-oxidation and the TCA cycle. Filled and open yellow circles indicate ¹³C and ¹²C, respectively. b-o, Relative quantities of ¹³C-labeled metabolites: β-oxidation intermediates (b-g), acetyl-CoA (h), and TCA cycle intermediates (i-o). Green arrows indicate the direction of metabolic flow. Data are presented as means±s.d. (WT n=3; Sqrdl^{ΔN/ΔN} n=3). *P<0.05, **P<0.01, ***P<0.001, determined by Student's t-test. p, Survival of Sqrdl^{ΔN/ΔN} mice fed a normal diet and those fed a high-fat diet. The log-rank test was used to determine statistical significance.

### FIG. 6. Sulfur Metabolome Analysis for SQR Mutant Mice.

a, Endogenous levels of sulfur metabolites in the liver, lung, and plasma of 3-week-old WT and Sqrdl^{ΔN/ΔN} mice. CysSH (cysteine), CysSSH (cysteine hydropersulfide), GSH (glutathione), GSSH (glutathione hydropersulfide), HS⁻ (hydrosulfide, or hydrogen sulfide anion), HSS⁻ (hydropersulfide, or hydrogen disulfide anion), H₂SO₃ (thiosulfate). Data are means±s.d. (WT n=4; Sqrdl^{ΔN/ΔN} n=4 or 5). *P<0.05, **P<0.01, ***P<0.001, determined by Student's t-test. b, Intracellular levels of sulfur metabolites in WT and Sqrdl^{ΔN/ΔN} MEFs. Data are means±s.d. (n=4). *P<0.05, **P<0.01 and ***P<0.001, determined by Student's t-test. c, Sulfur metabolites in mitochondria isolated from WT and Sqrdl^{ΔN/ΔN} MEFs with or without NaHS supplementation. The control consisted of a reaction buffer without mitochondria. Data are means±s.d. (n=3). *P<0.05, **P<0.01, ***P<0.001, determined by one-way ANOVA with Tukey's test. d, Flux analysis using ³⁴S-labeled cystine in WT and Sqrdl^{ΔN/AN} MEFs. ³⁴S-labeled sulfur metabolites were quantified at the indicated time points after addition of ³⁴S-labeled cystine to the culture media. Data are means±s.d. (n=3). **P<0.01, ***P<0.001, determined by two-way ANOVA with Tukey's test.

### FIG. 7. Whole-Cell Evaluation of SQR Contribution to Mitochondrial Activity.

a, Schematic illustration of JC-1 MEF staining, with representative confocal fluorescence images of JC-1-stained WT and Sqrdl^{ΔN/ΔN} MEFs. Scale bars, 50 m. b, Measurement of the OCR in WT and Sqrdl^{ΔN/ΔN} MEFs. The OCR was measured with the XF96 Extracellular Flux Analyzer. Data are means±s.d. (n=10). Rot, rotenone; AA, antimycin A; DNP, 2,4-dinitrophenol. c-e, Effects of the addition of cystine (c), NaHS (d), and Na₂S₂ (e) on mitochondrial membrane potential in WT and Sqrdl^{ΔN/ΔN} MEFs. f, Response of mitochondrial membrane potential to CATR in WT and Sqrdl^{ΔN/ΔN} MEFs. g, Effects of addition of Rot, atpenin A5 (AA5), AA, and CATR on mitochondrial membrane potential of WT MEFs. Data shown in c-g are means±s.d. (n=3 or 4). *P<0.05, **P<0.01, ***P<0.001, N.S. (not significant), determined by one-way ANOVA with Tukey's test.

### FIG. 8. Evaluation of the SQR Contribution to Mitochondrial Activity in Isolated Mitochondria.

a-c, The membrane potential of isolated mitochondria was monitored by means of TMRE fluorescence intensity. Mitochondria isolated from WT and Sqrdl^{ΔN/ΔN} MEFs were treated with NaHS (a) followed by treatments with succinate/glutamate/malate, Rot/AA5/AA, TMPD/AsA, and NaCN. Data are means±s.d. AsA, ascorbic acid. #P<0.0001, determined by two-way ANOVA with Tukey's test. (b,c) NaHS-induced membrane potential of mitochondria isolated from WT MEFs in the presence of 0.5 μM Rot/0.5 μM AA5 (b) and 0.5 μM AA (c). Data are means±s.d. d, Membrane potential induced by NaHS in mitochondria isolated from fibroblasts from a patient with Leigh syndrome. Data are means s.d. e-g, Representative oxygen consumption in response to sulfide by mitochondria isolated from mouse livers of WT mice (e) and Sqrdl^{ΔN/ΔN} mice (f); data are from three independent experiments. The OCR in response to NaHS is shown (g). Data are means±s.d. of average OCR induced by NaHS in three independent experiments. *P<0.05, determined by Student's t-test. h-j, Mitochondria isolated from WT and Sqrdl^{ΔN/ΔN} MEFs were treated with Na₂S₂ (h) followed by treatments with succinate/glutamate/malate, Rot/AA5/AA, TMPD/AsA, and NaCN. Data are means±s.d. #P<0.0001, determined by two-way ANOVA with Tukey's test. (i,j) Na₂S₂-induced membrane potential of mitochondria isolated from WT MEFs in the presence of 0.5 μM Rot/0.5 μM AA5 (i) and 0.5 μM AA (j). Data are means±s.d. k,l, NaHS-induced (k) and Na₂S₂-induced (1) membrane potential in mitochondria isolated from WT MEFs in the presence of 10 μM SS19, a hydrogen sulfide scavenger. Data are means±s.d. m, Membrane potential induced by GSSSG in mitochondria isolated from WT MEFs. Data are means±s.d. n, Sulfur metabolites in mitochondria isolated from WT and Sqrdl^{ΔN/ΔN} MEFs with or without GSSSG supplementation. Data are means±s.d. (n=3). *P<0.05, **P<0.01, ***P<0.001, determined by Student's t-test. a-d and h-n are representative results of more than three independent experiments. o, Schematic model of SQR-driven sulfur respiration in mitochondria. Hydropersulfides (shown as RSSₙH) synthesized by CARS2 are oxidized by SQR and transformed into oxidized polysulfides (R—Sₙ—R′), which are coupled with the generation of membrane potential. The resulting R—Sₙ—R′ is expected to accept electrons and protons from the ETC and the thioredoxin/thioredoxin reductase system (Trx/TrxR) in mitochondria. Q, ubiquinone; QH₂, ubiquinol; NADPH, nicotinamide adenine dinucleotide phosphate reduced; ΔΨm, membrane potential.

### REFERENCES

This section list references associated with the specification. The specification contains superscripts that are from 1 to 42. The entire disclosure each of the references is incorporated by reference regarding subjects discussed in sections/paragraphs where the superscripts are used.
1. Baross J A, Hoffman S E. Submarine hydrothermal vents and associated gradient environments as sites for the origin and evolution of life. Orig Life Evol Biosph 1985; 15(4):327-345.
2. Fisher C, Takai K, Le Bris N. (2007) Hydrothermal vent ecosystems. Oceanography 2007; 20(1):14-23.
3. Longchamp A, Mirabella T, Arduini A, MacArthur M R, Das A, Treviño-Villarreal J H, Hine C, Ben-Sahra I, Knudsen N H, Brace L E, Reynolds J, Mejia P, Tao M, Sharma G, Wang R, Corpataux J M, Haefliger J A, Ahn K H, Lee C H, Manning B D, Sinclair D A, Chen C S, Ozaki C K, Mitchell J R. Amino acid restriction triggers angiogenesis via GCN2/ATF4 regulation of VEGF and H₂S production. Cell 2018 Mar. 22; 173(1):117-129.e14.
4. Das A, Huang G X, Bonkowski M S, Longchamp A, Li C, Schultz M B, Kim L J, Osborne B, Joshi S, Lu Y, Treviño-Villarreal J H, Kang M J, Hung T T, Lee B, Williams E O, Igarashi M, Mitchell J R, Wu L E, Turner N, Arany Z, Guarente L, Sinclair D A.

Impairment of an endothelial NAD⁺-H₂S signaling network is a reversible cause of vascular aging. Cell 2018 Mar. 22; 173(1):74-89.e20.
5. Faller S, Hausler F, Goeft A, von Itter M A, Gyllenram V, Hoetzel A, Spassov S G. Hydrogen sulfide limits neutrophil transmigration, inflammation, and oxidative burst in lipopolysaccharide-induced acute lung injury. Sci Rep 2018 Oct. 2; 8(1):14676.
6. Osipov R M, Robich M P, Feng J, Liu Y, Clements R T, Glazer H P, Sodha N R, Szabo C, Bianchi C, Sellke F W. Effect of hydrogen sulfide in a porcine model of myocardial ischemia-reperfusion: comparison of different administration regimens and characterization of the cellular mechanisms of protection. J Cardiovasc Pharmacol 2009 October; 54(4):287-297.
7. Hine C, Mitchell J R. Calorie restriction and methionine restriction in control of endogenous hydrogen sulfide production by the transsulfuration pathway. Exp Gerontol 2015 August; 68:26-32. doi: 10.1016/j.exger.2014.
8. Ida T, Sawa T, Ihara H, Tsuchiya Y, Watanabe Y, Kumagai Y, Suematsu M, Motohashi H, Fujii S, Matsunaga T, Yamamoto M, Ono K, Devarie-Baez N O, Xian M, Fukuto J M, Akaike T. Reactive cysteine persulfides and S-polythiolation regulate oxidative stress and redox signaling. Proc Natl Acad Sci USA 2014 May 27; 111(21):7606-7611.
9. Akaike T, Ida T, Wei F Y, Nishida M, Kumagai Y, Alam M M, Ihara H, Sawa T, Matsunaga T, Kasamatsu S, Nishimura A, Morita M, Tomizawa K, Nishimura A, Watanabe S, Inaba K, Shima H, Tanuma N, Jung M, Fujii S, Watanabe Y, Ohmuraya M, Nagy P, Feelisch M, Fukuto J M, Motohashi H. Cysteinyl-tRNA synthetase governs cysteine polysulfidation and mitochondrial bioenergetics. Nat Commun 2017 Oct. 27; 8(1):1177.
10. Madigan M T, Brock T D. Photosynthetic sulfide oxidation by Chloroflexus aurantiacus, a filamentous, photosynthetic, gliding bacterium. J Bacteriol 1975 May; 122(2):782-784.
11. Weghe J G V, Ow D W. A fission yeast gene for mitochondrial sulfide oxidation. J Biol Chem 1999; 274:13250-13257.
12. Theissen U, Hoffmeister M, Grieshaber M, Martin W. Single eubacterial origin of eukaryotic sulfide:quinone oxidoreductase, a mitochondrial enzyme conserved from the early evolution of eukaryotes during anoxic and sulfidic times. Mol Biol Evol 2003; 20:1564-1574.
13. Rose P, Moore P K, Zhu Y Z. H₂S biosynthesis and catabolism: new insights from molecular studies. Cell Mol Life Sci 2017; 74:1391-1412.
14. Libiad M, Yadav P K, Vitvitsky V, Martinov M, Banerjee R. Organization of the human mitochondrial hydrogen sulfide oxidation pathway. J Biol Chem 2014 Nov. 7; 289(45):30901-30910.
15. Ackermann M, Kubitza M, Hauska G, Piña A L. The vertebrate homologue of sulfide-quinone reductase in mammalian mitochondria. Cell Tissue Res 2014 December; 358(3):779-792.
16. Lagoutte E, Mimoun S, Andriamihaja M, Chaumontet C, Blachier F, Bouillaud F. Oxidation of hydrogen sulfide remains a priority in mammalian cells and causes reverse electron transfer in colonocytes. Biochim Biophys Acta 2010 August; 1797(8):1500-1511.
17. Yong R, Searcy D G. Sulfide oxidation coupled to ATP synthesis in chicken liver mitochondria. Comp Biochem Physiol Part B 2001; 129:129-137.
18. Goubern M, Andriamihaja M, Niibel T, Blachier F, Bouillaud F. Sulfide, the first inorganic substrate for human cells. FASEB J 2007 June; 21(8):1699-1706.
19. Supekova L, Supek F, Greer J E, Schultz P G. A single mutation in the first transmembrane domain of yeast COX2 enables its allotopic expression. Proc Natl Acad Sci USA 2010 Mar. 16; 107(11):5047-5052.
20. Fujii S, Sawa T, Motohashi H, Akaike T. Persulfide synthases that are functionally coupled with translation mediate sulfur respiration in mammalian cells. Br J Pharmacol 2019 February; 176(4):607-615.
21. Landry A P, Ballou D P, Banerjee R. H₂S oxidation by nanodisc-embedded human sulfide quinone oxidoreductase. J Biol Chem 2017; 292:11641-11649.
22. Mimoun S, Andriamihaja M, Chaumontet C, Atanasiu C, Benamouzig R, Blouin J M, Tome D, Bouillaud F, Blachier F. Detoxification of H₂S by differentiated colonic epithelial cells: implication of the sulfide oxidizing unit and of the cell respiratory capacity. Antioxid Redox Signal 2012; 17:1-10.
23. Chandel N S, Budinger G R, Choe S H, Schumacker P T. Cellular respiration during hypoxia. Role of cytochrome oxidase as the oxygen sensor in hepatocytes. J Biol Chem 1997 Jul. 25; 272(30):18808-18816.
24. Yang C T, Wang Y, Marutani E, Ida T, Ni X, Xu S, Chen W, Zhang H, Akaike T, Ichinose F, Xian M. Data-driven identification of hydrogen sulfide scavengers. Angew Chem Int Ed Engl 2019 Aug. 5; 58(32):10898-10902.
25. Bednarski B, Andreesen J R, Pich A. In vitro processing of the proproteins GrdE of protein B of glycine reductase and PrdA of D-proline reductase from Clostridium sticklandii: formation of a pyruvoyl group from a cysteine residue. Eur J Biochem 2001 June; 268(12):3538-3544.
26. Fukawa T, Yan-Jiang B C, Min-Wen J C, Jun-Hao E T, Huang D, Qian C N, Ong P, Li Z, Chen S, Mak S Y, Lim W J, Kanayama H O, Mohan R E, Wang R R, Lai J H, Chua C, Ong H S, Tan K K, Ho Y S, Tan I B, Teh B T, Shyh-Chang N. Excessive fatty acid oxidation induces muscle atrophy in cancer cachexia. Nat Med 2016 June; 22(6):666-671.
27. Jain I H, Zazzeron L, Goldberger O, Marutani E, Wojtkiewicz G R, Ast T, Wang H, Schleifer G, Stepanova A, Brepoels K, Schoonjans L, Carmeliet P, Galkin A, Ichinose F, Zapol W M, Mootha V K. Leigh syndrome mouse model can be rescued by interventions that normalize brain hyperoxia, but not HIF activation. Cell Metab 2019 Oct. 1; 30(4):824-832.e3.
28. Krawchuk M D, Wahls W P. High-efficiency gene targeting in Schizosaccharomyces pombe using a modular, PCR-based approach with long tracts of flanking homology. Yeast 1999 Sep. 30; 15(13):1419-1427.
29. Gietz R D, Schiestl R H. High-efficiency yeast transformation using the LiAc/SS carrier DNA/PEG method. Nat Protoc 2007; 2(1):31-34.
30. Lai C Y, Jaruga E, Borghouts C, Jazwinski S M. A mutation in the ATP2 gene abrogates the age asymmetry between mother and daughter cells of the yeast Saccharomyces cerevisiae. Genetics 2002 September; 162(1):73-87.
31. Fabrizio P, Liou L L, Moy V N, Diaspro A, Valentine J S, Gralla E B, Longo V D. SOD2 functions downstream of Sch9 to extend longevity in yeast. Genetics 2003 January; 163(1):35-46.
32. Behringer, R. Manipulating the Mouse Embryo: A Laboratory Manual, Fourth edition. 2014; Cold Spring Harbor Laboratory Press, Cold Spring Harbor, NewYork.
33. Hashimoto M, Yamashita Y, Takemoto T. Electroporation of Cas9 protein/sgRNA into early pronuclear zygotes generates non-mosaic mutants in the mouse. Dev Biol 2016 1; 418(1):1-9.
34. Wei F Y, Zhou B, Suzuki T, Miyata K, Ujihara Y, Horiguchi H, Takahashi N, Xie P, Michiue H, Fujimura A, Kaitsuka T, Matsui H, Koga Y, Mohri S, Suzuki T, Oike Y, Tomizawa K. Cdk5rap 1-mediated 2-methylthio modification of mitochondrial tRNAs governs protein translation and contributes to myopathy in mice and humans. Cell Metab 2015 Mar. 3; 21(3):428-442.
35. Nishimura A, Shimauchi T, Tanaka T, Shimoda K, Toyama T, Kitajima N, Ishikawa T, Shindo N, Numaga-Tomita T, Yasuda S, Sato Y, Kuwahara K, Kumagai Y, Akaike T, Ide T, Ojida A, Mori Y, Nishida M. Hypoxia-induced interaction of filamin with Drpl causes mitochondrial hyperfission-associated myocardial senescence. Sci Signal 2018 Nov. 13; 11(556). pii: eaat5185.
36. Yuan M, Breitkopf S B, Yang X, Asara J M. A positive/negative ion-switching, targeted mass spectrometry-based metabolomics platform for bodily fluids, cells, and fresh and fixed tissue. Nat Protoc 2012; 7:872-881.
37. Smiley S T, Reers M, Mottola-Hartshom C, Lin M, Chen A, Smith T W, Steele G D Jr, Chen L B. Intracellular heterogeneity in mitochondrial membrane potentials revealed by a J-aggregate-forming lipophilic cation JC-1. Proc Natl Acad Sci USA 1991 May 1; 88(9):3671-3675.
38. Sherer T B, Trimmer P A, Parks J K, Tuttle J B. Mitochondrial DNA-depleted neuroblastoma (Rho°) cells exhibit altered calcium signaling. Biochim Biophys Acta 2000 Apr. 17; 1496(2-3):341-355.
39. Nicholls D G, Darley-Usmar V M, Wu M, Jensen P B, Rogers G W, Ferrick D A. Bioenergetic profile experiment using C2C12 myoblast cells. J Vis Exp 2010 Dec. 6; (46). pii:2511.
40. Scaduto R C Jr, Grotyohann L W. Measurement of mitochondrial membrane potential using fluorescent rhodamine derivatives. Biophys J 1999 January; 76(1 Pt 1):469-477.
41. Frezza C, Cipolat S, Scorrano L. Organelle isolation: functional mitochondria from mouse liver, muscle and cultured fibroblasts. Nat Protoc 2007; 2(2):287-295.
42. Ono K, Jung M, Zhang T, Tsutsuki H, Sezaki H, Ihara H, Wei F Y, Tomizawa K, Akaike T, Sawa T. Synthesis of L-cysteine derivatives containing stable sulfur isotopes and application of this synthesis to reactive sulfur metabolome. Free Radic Biol Med 2017 May; 106:69-79.

What is claimed is:

1. A method for improving mitochondrial functions, comprising:
a step for supplementing mitochondria with an efficient amount of a reactive persulfide, and
a step for increasing a membrane potential of mitochondria by said reactive persulfide.


SEQUENCE LISTING (10 sequences)

SEQ ID NO: 1 - protein, 19 aa, synthetic construct
  REGION 1..19: note = Synthetic
MAPLVTVVSSPRARLFACF

SEQ ID NO: 2 - DNA, 86 bp, synthetic construct
  misc_feature 1..86: note = Synthetic
  source 1..86: mol_type = other DNA
tgcttccttttagcctgatctatcctggtgatggccccactggtcactgttgctccagtccacgtgcccggctctttgcctgtttt

SEQ ID NO: 3 - DNA, 72 bp, synthetic construct
  misc_feature 1..72: note = Synthetic
  source 1..72: mol_type = other DNA
tgcttccttttagcctgatctatcactggtcactgttgtctccagtccacgtgcccggctctttgcctgttt

SEQ ID NO: 4 - DNA, 30 bp, synthetic construct
  misc_feature 1..30: note = Synthetic
  source 1..30: mol_type = other DNA
tgagttatcgactgaatataaacaccttat

SEQ ID NO: 5 - DNA, 50 bp, synthetic construct
  misc_feature 1..50: note = Synthetic
  source 1..50: mol_type = other DNA
ttaattaacccggggatccgttttagttgcaaaaatgcaaaagagaagtg

SEQ ID NO: 6 - DNA, 30 bp, synthetic construct
  misc_feature 1..30: note = Synthetic
  source 1..30: mol_type = other DNA
ttagtagcattttttctctagattctttta

SEQ ID NO: 7 - DNA, 50 bp, synthetic construct
  misc_feature 1..50: note = Synthetic
  source 1..50: mol_type = other DNA
gtttaaacgagctcgaattcagattcaatcatctttcttgcgtaaaataa

SEQ ID NO: 8 - DNA, 20 bp, synthetic construct
  misc_feature 1..20: note = Synthetic
  source 1..20: mol_type = other DNA
tatcctggtgatggccccac

SEQ ID NO: 9 - DNA, 22 bp, synthetic construct
  misc_feature 1..22: note = Synthetic
  source 1..22: mol_type = other DNA
tgcttccttttagcctgatcta

SEQ ID NO: 10 - DNA, 23 bp, synthetic construct
  misc_feature 1..23: note = Synthetic
  source 1..23: mol_type = other DNA
aaaacaggcaaagagccgggcac